(19) United States
(12) Patent Application Publication
(10) Pub. No.: US 2009/0131734 A1
(43) Pub. Date: May 21, 2009

(54) Implantable medical marker and methods of preparation thereof

(75) Inventors: David Maier Neustadter, Natania (IL); Giora Kornblau, Binyamina (IL)

Correspondence Address: MARTIN D. MOYNIHAN d/b/a PRTSI, INC., P.O. BOX 16446, ARLINGTON, VA, 22215, US

(73) Assignee: Navotek Medical Ltd., Yokneam (IL)

(21) Appl. No.: 11/791,890

(22) PCT Filed: Feb. 15, 2007
(86) PCT No.: PCT/IL2007/000214

§ 371 Date: May 30, 2007

Related U.S. Application Data
(60) Provisional application No. 60/773,931, filed on Feb. 16, 2006.
(60) Provisional application No. 60/773,930, filed on Feb. 16, 2006.
(60) Provisional application No. 60/804,178, filed on Jun. 8, 2006.

(30) Foreign Application Priority Data
Aug. 10, 2006 (IB) PCT/IB2006/052770
Aug. 10, 2006 (IB) PCT/IB2006/052771

(51) Int. Cl. A61M 36/12

(52) U.S. Cl. 600/8

(57) ABSTRACT

An implantable medical marker, the marker comprising a marker body adapted for insertion via a needle and adapted to define a volume with a smallest dimension larger than an inner diameter of the needle; and a radiation source—characterized by gamma emissions sufficient to exit the human body.

### RELATED APPLICATIONS

This application is a Continuation in part of International application PCT/IB2006/052771 filed on Aug. 10, 2006 entitled “Medical Treatment System and Method” and is also a Continuation in part of International Patent Application PCT IB2006/052770 filed on Aug. 10, 2006 and entitled “Localization of a Radioactive Source”.

This application also claims priority from U.S. Provisional Applications:
60/773,931 filed on Feb. 16, 2006, entitled “Radiation Oncology Application”;
60/773,930 filed Feb. 16, 2006, entitled “Localization of a Radioactive Source”;
60/804,178 filed on Jun. 8, 2006, entitled “Radioactive Medical Implants”;
The disclosures of these international and provisional applications are each fully incorporated herein by reference.

This application is related to:
PCT/IL2005/000871 filed on Aug. 11, 2005, entitled “Localization of a Radioactive Source within a Body of a Subject”; and
PCT/IL2005/001101 filed on Oct. 19, 2005; entitled “Tracking a Catheter Tip by Measuring its Distance From a Tracked Guide Wire Tip”.
U.S. Provisional Application 60/600,725 filed on Aug. 12, 2004, entitled “Medical Navigation System Based on Differential Sensor”;
U.S. Provisional Application 60/619,792 filed on Oct. 19, 2004, entitled “Using a Catheter or Guidewire Tracking System to Provide Positional Feedback for an Automated Catheter or Guidewire Navigation System”;
U.S. Provisional Application 60/619,897 filed on Oct. 19, 2004, entitled “Using a Radioactive Source as the Tracked Element of a Tracking System”;
U.S. Provisional Application 60/619,898 filed on Oct. 19, 2004, entitled “Tracking a Catheter Tip by Measuring its Distance from a Tracked Guide Wire Tip”;
U.S. patent application Ser. No. 11/463,664 filed on Aug. 10, 2006 and entitled “Medical Treatment System and Method”; and
U.S. patent application Ser. No. 11/463,659 filed on Aug. 10, 2006 and entitled “Medical Treatment System and Method”.
The disclosure of each of these applications is fully incorporated herein by reference.

### FIELD OF THE INVENTION

The invention relates generally to implantable markers which can be located using a tracking device and/or seen in a medical image.

### BACKGROUND OF THE INVENTION

Medical markers typically are either visible in medical imaging and/or emit a signal detectable by a dedicated detection device. In some cases, medical imaging provides a relative location of the marker with respect to an anatomic location, but does not provide an absolute location of the marker in terms of position co-ordinates. In other cases, a dedicated detection device provides an absolute location of the marker in terms of position co-ordinates but does not provide a relative position with respect to an anatomic location. Registration of relative and absolute location can be problematic.

Brachytherapy Seed Designs

In brachytherapy, ionizing radiation is applied to a target for therapeutic purposes by implantation of a brachytherapy “seed” which produces cytotoxic ionizing radiation. The seed is implanted within the body in proximity to the target.

U.S. Pat. No. 6,436,026 to Sioshani (RadioMed Corp.) and US 2004/0116767 by Lebovic disclose spiral configuration brachytherapy seeds. The Lebovic application discloses delivery of the seed via a needle. The disclosures of these applications are fully incorporated herein by reference.

WO 02/078785 by Radiovascular Inc.; WO 2004/026111 by Microsperix LLC.; U.S. Pat. No. 6,749,555 to Winkler (Proxima Therapeutics inc.); US 2003/0158515 by Gonzalez (Spiration Inc.) each disclose brachytherapy seed designs which anchor themselves within the body. The disclosures of these applications and patents are fully incorporated herein by reference.

Systems including Dedicated Detection Devices

U.S. Pat. No. 4,215,694 to Isakov teaches a device for tracking the position of an irradiated object and an electromechanical drive unit for aiming a beam source. The device for tracking the position relies upon sensors in the form of pulse transformers. The disclosure of this patent is fully incorporated herein by reference.

WO0154765 by ZMED teaches a system for aiming a radiation beam by aligning a frame (bed) holding a patient. The disclosure of this application is fully incorporated herein by reference.

Implantable Markers for Position Determination

US 2005/0261570 by Mate teaches implantation of excitable markers in/near a target. An external excitation source is then aimed at the marker to excite it. The excitation energy is used for position determination. Therapeutic radiation is aimed at a position determined by the marker excitation energy. The disclosure of this application is fully incorporated herein by reference.

US 2005/0027196 by Fitzgerald teaches a system for processing patient radiation treatment data. Fitzgerald teaches use of imaging equipment to determine positions of brachytherapy radiation sources implanted in a patient. The disclosure of this application is fully incorporated herein by reference.

WO 00/57923 teaches a radioactive seed which discloses the orientation and location of the seed when exposed to X-ray. Orientation is indicated by use of different radio-opaque materials. The disclosure of this application is fully incorporated herein by reference.

US 2005/0197564 by Dempsey teaches use of MRI to identify where tracer is taken up, as ionizing radiation is applied. The disclosure of this application is fully incorporated herein by reference.

A series of US patents assigned to Calypso Medical Technologies (e.g. U.S. Pat. No. 6,977,504; U.S. Pat. No. 6,889,833; U.S. Pat. No. 6,838,990; U.S. Pat. No. 6,822,570 and U.S. Pat. No. 6,812,842) describe use of AC electromagnetic localization transponders in conjunction with a position determination system. The disclosures of these patents are fully incorporated herein by reference.

### Location Determination by Monitoring Intrabody Radiation

Co-pending PCT application WO 2006/016368 by the inventors of the present invention teaches the use of multiple directional sensors for real time measurement of the 3 dimensional position of a gamma emitting source. The disclosure of this application is fully incorporated herein by reference.

U.S. Pat. No. 6,603,124 to Maublant teaches the use of a directional sensor for detecting a direction towards a gamma emitting source. The disclosure of this patent is fully incorporated herein by reference.

Markers Visible in Medical Imaging

Soft tissue markers which can be visualized using medical imaging are described in, for example, U.S. Pat. Nos. 6,575,991; 6,228,055; 6,425,903; 6,056,700; 6,234,177; 6,181,960; 6,662,041 and 6,862470. The disclosure of each of these patents is fully incorporated herein by reference. The list does not purport to be exhaustive. Markers which can be visualized using medical imaging can be constructed of metal and/or polymers and/or gels. Various markers described in these patents are visible in one or more of Ultrasound, X-ray, CT, and MRI images. Some of these markers can be implanted through very narrow needles (25 gauge or narrower), although most of them are designed for implantation through large bore biopsy needles (13 or 14 gauge). Some of these markers have specific features intended to reduce migration, such as bioadhesive material and/or mechanical hook and/or mechanical flexibility.

Implantable Brachytherapy Sources

U.S. Pat. Nos. 6,132,359; 5,713,828; 5,997,463; 6,419,621; 6,986,880; 6,575,888 and 7,083,566 describe implants made of metal, polymers, and micro sphere containing gels with radioactive material incorporated into the implant. The disclosure of each of these patents is fully incorporated herein by reference. The list does not purport to be exhaustive. Many of these patents describe implants with features intended to reduce migration and to increase visibility in one or more medical imaging modalities. The described markers include a cyto-toxic amount of radiation intended for therapy (i.e. brachytherapy). As a group, implants described in these patents are adapted to provide high levels of localized radiation in a form which is absorbed by adjacent tissue with only a small amount of radiation reaching distant organs or escaping the body.

These implanted sources typically rely upon beta emissions or low energy (below 100 keV) gamma emissions. The energy from low energy gamma emissions is primarily absorbed within a few centimeters as they pass through soft tissue. The energy from beta emissions is primarily absorbed within a few millimeters as they pass through soft tissue. The absorption transfers the emitted energy to the tissue and produces a local cytotoxic effect without exposing the rest of the patient's body and/or other people to ionizing radiation. As a result, these implanted sources are generally not well suited for detection, location determination or tracking by a radiation sensor or detector located outside the body.

Vaso-Occlusive Devices

U.S. Pat. No. 6,616,591 describes a radioactive polymer which can be used together with vaso-occlusive devices to render them radioactive. This patent describes use of the polymers in conjunction with devices designed for insertion into vessels within the body via a catheter. The disclosure of this patent is fully incorporated herein by reference.

### SUMMARY OF THE INVENTION

An aspect of some embodiments of the invention relates to an implantable medical marker adapted for improved visibility in a medical image. In an exemplary embodiment of the invention, the marker includes a radioactive source. In an exemplary embodiment of the invention, visibility is improved by expansion of the marker as, or after, it exits an implantation tool. Optionally, this expansion anchors contributes to a reduction in a tendency of the marker to migrate.

Optionally, the radioactive source has a clinically insignificant effect on surrounding tissue but produces a detectable radioactive signal outside the body. In an exemplary embodiment of the invention, the detectable radioactive signal comprises at least 400,000 emitted photons per second that escape the patient's body. In an exemplary embodiment of the invention, the detectable radioactive signal is used for location determination and/or tracking.

In an exemplary embodiment of the invention, the marker is adapted for injection via a narrow needle. In an exemplary embodiment of the invention, the narrow needle is a 29, 27, 25, 23 or 20 gauge or intermediate or narrower or wider gauge. In an exemplary embodiment of the invention, the marker is configured and/or formed of suitable materials so as to be visible in a medical image such as, for example an X-ray image, a CT image, an MRI image or an ultrasound image.

In an exemplary embodiment of the invention, the marker is compressed to conform to an internal volume of the injection needle and expands to define a larger volume when injected into soft tissue. In an exemplary embodiment of the invention, the defined larger volume is filled to a sufficient degree by the marker to make the defined larger volume visible in the medical image. Optionally, filling to a sufficient degree refers to 5, 10, 15, 25 or 50% or lesser or intermediate or larger percentages of the defined volume.

In an exemplary embodiment of the invention, the implantable radioactive medical marker is adapted for implantation via a 20 gauge or narrower tool (e.g. needle) and further adapted to define one or more three dimensional volumes having a smallest dimension of at least 1 mm after implantation in tissue. Optionally, the combination of implantation via a 20 gauge or narrower needle and definition of a volume having a smallest dimension of at least 1 mm after implantation in tissue requires that the marker expand and/or change shape and/or deform upon implantation. Optionally, the three dimensional volume(s) defined by the marker upon implantation in tissue comprise a 2-5 mm spheroid or ellipsoid.

In an exemplary embodiment of the invention, the marker comprises a chain which folds on itself upon implantation to define a 2-5 mm spheroid. Optionally, the spheroid comprises a plurality of shapes, optionally irregular shapes. In an exemplary embodiment of the invention, the shapes increase the visibility of the implanted marker in a selected imaging mode. Optionally, the shapes contribute to an increase in surface area which contributes to a reduction in a tendency to migrate within a tissue. Alternatively, or additionally, an increase in surface area increases visibility of the marker in ultrasound imaging. Optionally, the marker is formed of a material and/or constructed so that it does not cause artifacts in an MRI image.

In an exemplary embodiment of the invention, the marker has a relatively uniform spatial distribution of radioactivity. Alternatively or additionally, the implanted marker has a radio-opaque aspect of sufficient size and density to be visible in X-ray and/or CT imaging in all orientations. “Radio-opaque” as used in this specification and the accompanying claims indicates absorption of at least 1% of an incident X-ray beam. Optionally, exemplary markers according to the invention achieve 1, 2, 5, 10 or 15% absorption or intermediate or greater levels of absorption.

An aspect of some embodiments of the invention relates to use of a selective shield on an implantable medical marker containing a source of ionizing radiation. In an exemplary embodiment of the invention, the selective shield impedes transmission of beta particle to a greater degree than it impedes transmission of gamma particles. In an exemplary embodiment of the invention, the selective shield significantly reduces the quantity of beta-particle exiting the marker. In an exemplary embodiment of the invention, the shield substantially prevents any beta radiation from exiting the marker.

A broad aspect of the invention relates to the formation of a radioactive marker via the incorporation of radioactive micro-spheres in a biocompatible amorphous mass. Optionally the micro-spheres are provided as an aliquot. Injection of the fluid amorphous mass at a desired location optionally produces a radioactive implant with reduced migration.

For purposes of this specification and the accompanying claims, the term “amorphous” means having no fixed form at the time it is introduced into the body. Optionally, the amorphous mass may harden, set and/or solidify after injection.

For purposes of this specification and the accompanying claims, the terms “disperse” and “dispersal” mean motion of micro-spheres away from a center of mass of the amorphous mass.

In an exemplary embodiment of the invention, the marker is implanted as a contiguous unit. Optionally, the contiguous unit remains contiguous (e.g. does not fall apart) for 7, 14, 21, 42 or 84 days or lesser or intermediate or greater times.

In an exemplary embodiment of the invention, migration of less than 10 mm, optionally, less than 5 mm, optionally less than 2 mm, optionally less than 1 mm or intermediate or lesser values of the marker is achieved. Optionally, the migration is measured for the duration of a medical procedure in which the marker is employed. Medical procedure times may be, for example, 2, 4, 8 or 12 weeks or lesser or intermediate or greater times.

In an exemplary embodiment of the invention, the amorphous mass comprises one or more of a glue, a cement and/or a soft matrix. Optionally, these materials may function as carriers, diluents or excipients. Optionally, other carriers and/or diluents and/or excipients are included in the amorphous mass.

In an exemplary embodiment of the invention, the soft matrix induces tissue-in-growth. Optionally, the soft matrix decays within the body over time. Optionally, decay of the soft matrix does not cause migration and/or dispersal of a clinically significant amount of radio-isotope. Optionally, decay of the soft matrix does not influence subsequently acquired images.

An aspect of some embodiments of the invention relates to use of a gamma emitting radioisotope in a coil shaped implantable medical device. Optionally, the coiled configuration reduces migration. Optionally, the coiled shape has substantially no elastic memory.

In an exemplary embodiment of the invention, the radioactive source is supplied as an approximately spherical adhesive drop with a diameter of between 2.0 and 5.0 mm.

In an exemplary embodiment of the invention, the marker includes a fixation element integrally formed with or attached to the radiation source. Optionally, the fixation element is adapted to prevent migration and/or unwanted dispersal of the source within the body. Optionally, the fixation element employs a physical configuration and/or an adhesive material and/or a coating to make the source self anchoring.

Optionally, the marker includes a radio-opaque portion. In an exemplary embodiment of the invention, the radio-opaque portion allows visualization of the marker using X-ray based imaging methods. Optionally, visualization is useful during placement of the marker near a target.

An aspect of some embodiments of the present invention relates to a kit including an implantable marker as described above together with an implantation needle adapted to contain the marker and an ejection tool adapted to expel the marker from the injection needle. In an exemplary embodiment of the invention, the marker is inserted into the implantation needle at a manufacturing facility. Optionally, the ejection tool is inserted into the implantation needle at a manufacturing facility.

For purposes of this specification and the accompanying claims, the terms “migrate” and “migration” mean shifting of a center of mass of the radioactive marker relative to a defined location in the surrounding tissue. Optionally, migration is measured only after a first image has been acquired or a first measurement has been made. The phrase “additional migration” indicates migration which occurs after a selected period of time after placement of the marker.

As an illustrative example, if a marker containing a radio-opaque element and a radiation source is implanted a week before it is intended to be used as a marker during a medical treatment, the marker may be subject to migration prior to the treatment. Optionally, this migration ceases or becomes negligible as a result of setting and/or hardening and/or adhesion and/or tissue in-growth. Prior to beginning the treatment, an image is acquired (e.g. X-ray or CT) which shows the relative positions of the centers of mass of the marker and the target. These relative positions may then be used to calculate the target position based on the marker position. In an exemplary embodiment of the invention, little or no additional migration occurs after this stage and throughout the treatment.

According to various exemplary embodiments of the invention, implantable radioactive markers as described above are employed in one or more of tracking of items, mapping, aiming of external devices, monitoring of tumor and/or therapy progression, and positioning a target with respect to an external reference frame.

In an exemplary embodiment of the invention, expansion of the marker as it exits an injection tool contributes to an ability to use a narrower gauge tool and/or to improved marker visibility after implantation and/or to reduced radiation dose to the tissue surrounding the marker.

The term “expansion” as used in this specification and the accompanying claims includes, but is not limited to, deformation and/or reshaping. In an exemplary embodiment of the invention, the marker expands in an irregular fashion to a disorganized form which defines a volume.

The term “disorganized” as used in this specification and the accompanying claims indicates one or more of disordered, chaotic and irregular. Optionally, two identical markers injected into similar tissue are characterized by non-identical disorganized forms after expansion.

Optionally, a marker includes one or more disorganized portions prior to insertion. In an exemplary embodiment of the invention, these disorganized portions are compressed during insertion into a needle.

In an exemplary embodiment of the invention, there is provided an implantable medical marker, the marker comprising:

(a) a marker body adapted for insertion via a needle and adapted to define a volume with a smallest dimension larger than an inner diameter of the needle; and
(b) a radiation source—characterized by gamma emissions sufficient to exit the human body.

Optionally, the smallest dimension is at least 1 mm.

Optionally, the gamma emissions produce between 1×10⁵ and 3×10⁸ photons/second.

Optionally, the gamma emissions produce not more than 5×10⁷ photons/second.

Optionally, the gamma radiation is characterized by an average energy of at least 50 kev.

Optionally, the gamma radiation is characterized by an average energy of at least 150 kev.

Optionally, the gamma radiation is characterized by an average energy not exceeding 400 kev.

Optionally, the gamma radiation is characterized by an average energy not exceeding 1000 kev.

Optionally, the marker is characterized by at least 1% absorption of an incident X-ray beam on the defined volume.

Optionally, the marker produces a radiation dose not exceeding 100 Gy at a distance of 2 mm from the marker in 6 months.

Optionally, the marker produces a radiation dose not exceeding 40 Gy at a distance of 2 mm from the marker in 6 months.

Optionally, marker body includes one or more disorganized sections.

Optionally, the defined volume is only partially occupied by the marker body.

Optionally, the marker body is disorganized.

Optionally, the marker body is jumbled.

Optionally, the marker body is random.

Optionally, the marker body is chaotic.

Optionally, a center of opacity and a center of radioactivity are both within the defined volume.

Optionally, a center, of opacity and a center of radioactivity are in a defined spatial relationship with respect to one another.

Optionally, a center of opacity and a center of radioactivity are spaced apart less than 20% of the largest dimension of the defined volume.

Optionally, the marker is characterized by a spherically uniform distribution of radiation emission within 30%.

Optionally, the marker is characterized in that at least a portion of the marker is adapted to absorb between 2 and 25 percent of 70 kev X-ray radiation incident on the marker.

Optionally, the marker is adapted for insertion via a needle a 21 gauge or narrower needle.

Optionally, the marker body is adapted for insertion via a needle a 23, 25 or 27 gauge or narrower needle.

Optionally, the marker body is a volume of non-solid material.

Optionally, the volume of non-solid material includes micro-spheres which promote tissue in-growth.

Optionally, the non-solid material is selected from the group consisting of a gel, a glue and a cement.

Optionally, the non-solid material is bio-absorbable.

Optionally, the radiation source comprises radioactive micro-spheres mixed into the non-solid material.

Optionally, the micro-spheres are characterized by a degree of radio-opacity which contributes to a visibility of the marker in the X-ray based imaging mode.

Optionally, the volume of non-solid material includes micro-spheres which are characterized by a degree of radio-opacity which contributes to a visibility of the marker in the X-ray based imaging mode.

Optionally, the non-solid material includes the radiation source.

Optionally, the non-solid material is characterized by a degree of radio-opacity which contributes to a visibility of the marker in an X-ray based imaging mode.

Optionally, the marker body is constructed of a radio-opaque radioactive metal.

Optionally, the marker body comprises a radio-opaque radioactive wire with a spring-like memory.

Optionally, the marker body comprises a coil.

Optionally, the coil includes a Platinum/Iridium alloy.

Optionally, the coil is adapted for folding.

Optionally, the coil comprises a selective shield adapted to significantly reduce beta emissions from the marker.

Optionally, the selective shield comprises a material chosen from a heavy metal and a plastic.

Optionally, the selective shield comprises at least one material selected from platinum and gold.

Optionally, the selective shield is characterized by a thickness of 0.025 to 0.25 mm.

Optionally, the marker body comprises a plurality of radioactive beads in a flexible sleeve.

Optionally, each radioactive bead includes a 0.025 to 0.250 mm thick layer of a beta radiation shielding material.

Optionally, the beads are characterized by a shape selected from among spherical and cylindrical.

Optionally, the marker body comprises a chain of beads connected by flexible wire.

Optionally, the beads comprise a radioactive radio-opaque material.

Optionally, the beads comprise a radio-opaque material surrounding a radioactive core.

Optionally, a radio-opaque material and the radiation source are each encapsulated within the beads.

Optionally, the beads comprise a beta shielding material adapted to selectively dampen beta emissions from the radiation source.

Optionally, the beads are characterized by a thickness of 0.025 to 0.25 mm.

Optionally, the marker body is adapted to remain contiguous.

In an exemplary embodiment of the invention, there is provided kit comprising a marker as described and an insertion tool.

Optionally, the kit comprises an ejection tool.

Optionally, the insertion tool comprises a needle.

Optionally, the implanted marker is provided loaded into the insertion tool.

In an exemplary embodiment of the invention, there is provided a method of preparing an implantable medical marker, the method comprising:

(a) inserting a plurality of radioactive beads into a sleeve; and
(b) inducing the sleeve to contract.

Optionally, the inducing includes at least one action selected from the group consisting of stretching, heating and chemically treating the sleeve.

Optionally, the contraction forms narrowed portions between the beads.

In an exemplary embodiment of the invention, there is provided a method of preparing an implantable medical marker, the method comprising:

(a) associating an amount of gamma radiation with a metal wire;
(b) forming the wire into a desired shape; and
(c) inserting the wire in a needle.

In an exemplary embodiment of the invention, there is provided a method of producing a non-migrating radioactive marker in situ in a subject, the method comprising:

(a) preparing an aliquot of radio-labeled microspheres; and

(b) injecting the aliquot at a desired location.

In an exemplary embodiment of the invention, there is provided an injectable pharmaceutical composition for formation of a non-migrating radioactive implant, the pharmaceutical composition comprising:

(a) an active ingredient including an aliquot of radioactive microspheres, and

(b) carriers, diluents and excipients.

In an exemplary embodiment of the invention, there is provided an implantable medical marker; comprising:

(a) a plurality of radioactive microspheres; and
(b) a biocompatible amorphous mass including said microspheres;

wherein the amorphous mass is adapted to prevent dispersion of the radioactive microspheres within the body.

### BRIEF DESCRIPTION OF THE DRAWINGS

Exemplary non-limiting embodiments of the invention are described in the following description, read with reference to the figures attached hereto. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1 is a simplified flow diagram of a method according to an exemplary embodiment of the invention;

FIG. 2a is a side view of an implantable medical marker according to an exemplary embodiment of the invention;

FIG. 2b is a lateral cross section of an implantable medical marker according to the exemplary embodiment of FIG. 2A loaded into an implantation tool;

FIG. 3a is a side view of an implantable medical marker according to an exemplary embodiment of the invention;

FIG. 3b is a lateral cross section of an implantable medical marker according to the exemplary embodiment of FIG. 2C loaded into an implantation tool;

FIGS. 4a and 4b are side views of exemplary embodiments of injection tools suitable for use in injection of bioadhesive materials according to some embodiments of the invention;

FIGS. 5, 6, 7, 8, 9 10 and 11 are side views of implantable medical markers according to different exemplary embodiments of the invention;

FIG. 12a is a side view of an implantable medical marker according to an exemplary embodiments of the invention;

FIGS. 12b, 12c and 12d are as series of views illustrating assembly of an implantable medical marker of the type depicted in FIG. 12a;

FIG. 13a is a side view of an implantable medical marker according to an exemplary embodiments of the invention; and

FIGS. 13b, 13c, 13d and 13e are as series of views illustrating assembly of an implantable medical marker of the type depicted in FIG. 13a.

FIG. 14 is a schematic representation of an exemplary helical marker body illustrating exemplary dimensions.

### DESCRIPTION OF EXEMPLARY EMBODIMENTS

### Overview

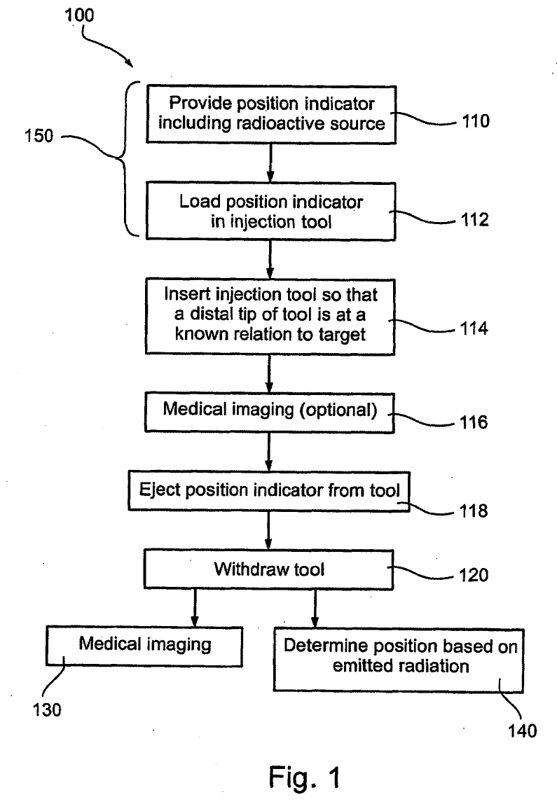

FIG. 1 is a simplified flow diagram of an implantation procedure 300 according to an exemplary embodiment of the invention.

At 110 an implantable marker including a radioactive source adapted to function as a marker is provided. According to some exemplary embodiments of the invention, the marker comprises a wire. Optionally, the marker is longer than an injection tool into which it will be loaded for injection. In an exemplary embodiment of the invention, the wire is coiled, folded or disorganized (e.g. jumbled) and then compressed to facilitate loading into the injection tool. Optionally, a degree of coiling folding or jumbling varies along an axial length of the marker. In an exemplary embodiment of the invention, disorganized sections are axially distributed along the marker at intervals, optionally regular intervals.

At 112, the marker is loaded into an injection tool. 150 indicates that 110 and 112 may optionally be performed at a manufacturing facility so that the marker is provided as an individually wrapped sterilized unit loaded into an injection tool. Alternatively, the marker and injection tool can be provided as a kit so that loading 112 is performed just prior to insertion 114.

At 114, the injection tool is inserted so that a distal tip of the tool is at a known displacement from the target. Optionally the known displacement is small and the distal tip of the tool approaches a boundary of the target. Optionally the distal tip of the tool is within the target.

116 indicates that insertion 114 may optionally be guided and/or evaluated by medical imaging. Guidance for placement and/or post placement evaluation of relative positions of the marker and the target may be conducted, for example, by ultrasound, fluoroscopy, standard X-ray imaging, CT, MRI or any other available imaging means.

At 118, the marker is ejected from the injection tool. Optionally, ejection is a process whereby the indicator is advanced as the needle is retracted. Optionally, the needle is held in place as a stylet inserted into the needle ejects the marker. Optionally, ejection is at a location which has been evaluated by imaging 116. In an exemplary embodiment of the invention, the marker expands to define a volume whose smallest dimension is larger than an inner diameter of the injection tool (e.g. needle) at this stage.

At 120, the injection tool is withdrawn. In an exemplary embodiment of the invention, medical imaging 130 and/or determination of position 140 based upon emitted radiation are performed before and/or after withdrawal 120 of the injection tool. Optionally, imaging 130 and imaging 116 are performed using a same, or a different, imaging modality (e.g. X-ray, CT, MRI or Ultrasound).

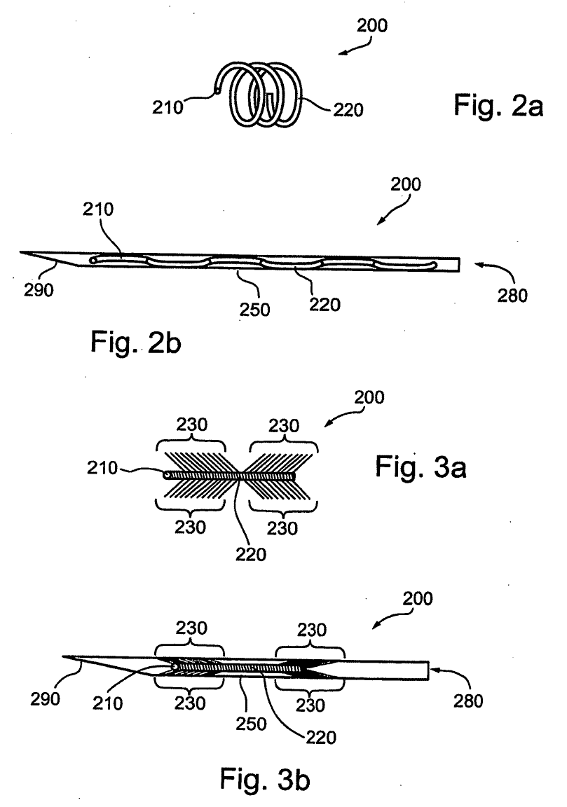

FIGS. 2a, 2b 3a and 3d illustrate provision 110 and loading 112 in the context of a coiled (FIGS. 2a and 2b) and a herring bone (FIGS. 2c and 2d) exemplary configuration of implantable markers 200 according to exemplary embodiments of the invention.

FIGS. 2A and 3a are schematic representations of implantable markers according to exemplary embodiments of the invention. In the pictured exemplary embodiments, marker 200 comprises a radioactive source 210 and a radio-opaque portion 220. Optionally, radio-opaque portion 220 serves as a fixation element. Optionally, additional anchoring structures 230 (FIG. 3a) are included. In an exemplary embodiment of the invention, marker 200 is coated with a biocompatible coating. Optionally, the coating renders marker 200 inert with respect to the body. In an exemplary embodiment of the invention, implantation of marker 200 does not elicit an immune and/or inflammatory response.

An exemplary embodiment depicted in FIG. 2a illustrates a spiral configuration. Optionally, the spiral configuration serves to anchor marker 200 in the body after it is deployed at a desired location. In an exemplary embodiment of the invention, the spiral is characterized by an elastic memory so that it tends to resume its spiral shape. In an exemplary embodiment of the figure, radio-opaque portion 220 is configured as a spiral and radioactive source 210 is concentrated at one end of marker 200. In additional exemplary embodiments of the invention, radioactive source 210 may be concentrated in a different location with respect to the spiral or diffused along the spiral.

In an exemplary embodiment of the invention, the radioactive material is positioned so that the radioactive emissions are substantially spherically uniform.

In an exemplary embodiment, depicted in FIG. 3a, a straight configuration is illustrated. Optionally, a herringbone pattern of filaments 230 characterized by an elastic memory (or super-elastic, or shape-memory) serves to anchor marker 200 in the body after it is deployed at a desired location. In the exemplary embodiment of the figure, radio-opaque portion 220 is configured as a straight cylinder and radioactive source 210 is concentrated at one end of marker 200. In additional exemplary embodiments of the invention, radioactive source 210 may be concentrated in a different location with respect to the cylinder or diffused along the cylinder. In an exemplary embodiment of the figure, radioactive source 210 may be a radioactive coating over a non-radioactive material.

FIGS. 2B and 3b are schematic representations of the markers according to exemplary embodiments of the invention depicted in FIGS. 2A and 3a respectively loaded in an injection needle 250. In an exemplary embodiment of the invention, needle 250 is a standard hypodermic needle, for example a 20 to 25 gauge needle.

FIG. 2B illustrates the compression of spiral portion 220 to a kinked straight configuration within needle 250.

FIG. 3b illustrates the compression of the herringbone pattern of filaments 230 within a needle 250. Application of an ejection force (e.g. from an inserted ejection tool) from proximal side 280 causes ejection of source 200 from distal aperture 290. Elastic memory of relevant portions of source 200 causes the ejected source to tend to revert to the relevant uncompressed configuration. In an exemplary embodiment of the invention, an ejection force is supplied by an ejection tool and/or by a stream of liquid.

### Exemplar Non-Solid Markers

In some exemplary embodiments of the invention, the implantable marker is provided as a non-solid marker. Optionally, the non-solid marker comprises an amorphous mass. In other exemplary embodiments of the invention, the implantable marker comprises both solid and non-solid components. The phrase “non-solid” as used in this specification and the accompanying claims includes liquids and/or gels as well as liquid or gel based slurries with solid particles (e.g. micro-spheres) suspended therein. Optionally, the non-solid material sets after injection or remains non-solid after injection. Optionally, the non-solid material comprises one or more of a glue, an adhesive a cement and an emulsion. Optionally, all or part of the non-solid material is bio-absorbable.

In an exemplary embodiment of the invention, the implantable marker comprises a droplet of biocompatible glue which contains a desired radioactive isotope. Optionally, the adhesive properties of the droplet reduce a tendency to migrate or shift after injection. Optionally, the adhesive drop is contiguous and/or non-dispersing. Optionally, the droplet also includes radio-opaque material. According to this exemplary embodiment of the invention, it is the marker itself which adheres strongly to the surrounding tissue without benefit of a separate physical anchor (e.g. spiral 220 or filaments 230). In an exemplary embodiment of the invention, a large (2-5 mm in diameter or intermediate diameters) biocompatible glue droplet, optionally including radio-opaque material can be injected through a narrow (20-27 gauge or intermediate gauge) needle since the glue is in a liquid or gel state at the time of injection. Optionally, the source is biodegradable and begins to lose integrity to a significant degree after 8-12 weeks. Optionally, the marker is metabolized and the radioisotope contained therein is excreted from the body. Optionally, the radioisotope particles within the glue droplet are individually coated with a biocompatible material so that they remain biocompatible as the glue degrades and the particles disperse and are excreted from the body. Optionally, the glue droplet is injected in a liquid or semi-liquid state and sets to a solid mass after injection. In an exemplary embodiment of the invention, the amount of radioactivity per unit volume is adjusted according to the specific application.

Biocompatible glues suitable for use in the context of exemplary embodiments of the invention are commercially available and one of ordinary skill in the art will be able to select a suitable glue for a contemplated exemplary embodiment. Examples of biocompatible glues include, but are not limited to, Omnex (Closure Medical Corporation, Raleigh, N.C.) and BioGlue (Cryolife, Atlanta, Ga.).

According to various exemplary embodiments of the invention, the biocompatible glue may be a two-component glue (e.g. BioGlue, Cryolife, Atlanta, Ga.; USA) or a one-component glue which hardens upon contact with human tissue (e.g. Omnex, Closure Medical Corporation, Raleigh, N.C.; USA), or a glue that is hardened by the application of a transformation energy (e.g. UV light; heat; or ultrasound).

In an exemplary embodiment of the invention, a radioactive source comprising a droplet of biocompatible glue which contains a desired radioactive isotope is provided as part of a kit including an injection tool. Optionally, the injection tool mixes glue components as the glue is being injected.

In an exemplary embodiment of the invention, the injection tool is a transparent syringe marked with a scale so that the amount of glue injected is readily apparent to an operator. Optionally, the scale is marked in volume and/or drop diameter and/or radioactivity. In an exemplary embodiment of the invention, there is a knob, slider, or other mechanical actuator on the injection tool which can be positioned to a certain volume, drop diameter, or radioactivity marking which causes the appropriate amount of glue to be injected when the injection tool is activated. In an exemplary embodiment of the invention, the injection tool includes an inflatable balloon at the end of the applicator to create a space in the tissue for the bead of glue to fill. Optionally, the injection tool applies a transformation energy.

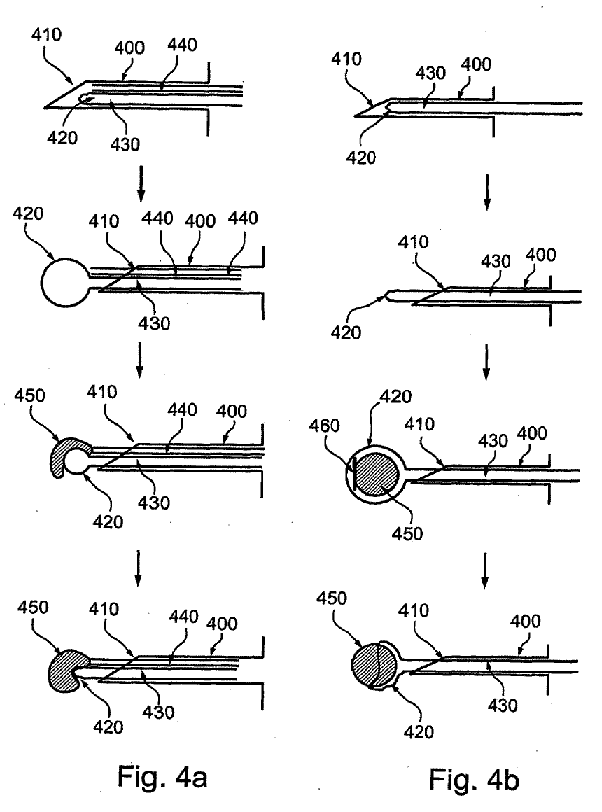

FIG. 4A illustrates one exemplary embodiment of an injection tool including two hollow tubes 430 and 440 within a needle 400. In this exemplary embodiment, tube 430 is fitted with an inflatable balloon 420 at its distal end and tube 440 is open at its distal end. Optionally, after insertion, needle 400 is retracted slightly so tubes 430 and 440 extend beyond distal end 410 of needle 400. Balloon 420 is then inflated to create a hole in tissue in or near a target. Inflation may be, for example, with a physiologically compatible gas (e.g., oxygen, Nitrogen or an oxygen containing mixture) or a fluid (e.g. sterile saline). According to this exemplary embodiment, as balloon 420 is deflated, bioadhesive material 450 containing a radioisotope is concurrently injected through tube 440 to fill the void left by deflating balloon 420. Optionally, the radioisotope is dispersed within bioadhesive material 450. Optionally, material 450 includes a radio-opaque material. In an exemplary embodiment of the invention, partially hardened bioadhesive 450 adheres to the surrounding tissue.

FIG. 4B illustrates an additional exemplary embodiment of an injection tool which employs a single hollow tube 430 within a needle 400. The figure illustrates an exemplary sequence of events from top to bottom. In this exemplary embodiment, tube 430 is fitted with an inflatable balloon 420 at its distal end. Optionally, after insertion, needle 400 is retracted slightly so tube 430 extends beyond distal end 410 of needle 400. Balloon 420 is then inflated. In this exemplary embodiment, inflation is by filling the balloon with bioadhesive material 450 containing a radioisotope. Optionally, the radioisotope is dispersed within bioadhesive material 450. Optionally, material 450 includes a radio-opaque material. Optionally, a wire 460 incorporated into balloon 420 is heated, optionally by an electric current. In an exemplary embodiment of the invention, heating of wire 460 melts at least a portion of balloon 420 near the wire. Optionally, this melting allows balloon 420 to be retracted into needle 400. In an exemplary embodiment of the invention, partially hardened bioadhesive 450 adheres to the surrounding tissue.

In an exemplary embodiment of the invention, bioadhesive material 450 includes radioactive micro-spheres.

In an exemplary embodiment of the invention, micro-spheres including a radioisotope are prepared. Injection of the micro-spheres, optionally as part of a biocompatible fluid amorphous mass produces a radioactive marker which does not migrate. In an exemplary embodiment of the invention, use of micro-spheres reduces an immune response and/or reduces migration and/or dispersal of the isotope.

Optionally, micro-spheres characterized by a diameter of 25 microns or more tend to resist phagocytosis. Optionally, the micro-spheres are coated with or constructed from, materials which induce tissue in-growth (e.g. Calcium Hydroxylapatite). The tissue in-growth can reduce migration and/or dispersal.

Optionally, a roughened surface of the micro-spheres reduces migration and/or dispersal by increasing friction between the micro-spheres and surrounding tissue.

Optionally, a smooth surface of the micro-spheres reduces migration and/or dispersal by reducing an inflammatory and/or immune response to the micro-spheres.

In an exemplary embodiment of the invention, the amorphous mass comprises, optionally consists essentially of, a glue containing radioactive micro-spheres for example commercially available biocompatible glue. The term “glue”, as used in this specification and the accompanying claims indicates a material which sets to a mass which adheres to adjacent biological tissue.

In an exemplary embodiment of the invention, the amorphous mass comprises, optionally consists essentially of, a cement containing radioactive micro-spheres. Optionally, a calcium phosphate based cement is employed. Optionally, the calcium phosphate becomes calcium hyroxylapatite after injection. The term “cement”, as used in this specification and the accompanying claims indicates a material which sets to a mass which is non-adhesive with respect to soft tissue. In an exemplary embodiment of the invention, the cement comprises calcium phosphate, calcium hydroxylapatite or PMMA.

Optionally, materials which are approved for injection into the body such as calcium phosphate and/or calcium hyroxylapatite are employed in preparation of the marker. Optionally, the radioactive micro-spheres themselves are made of, or coated with, calcium phosphate and/or calcium hydroxylapatite. These materials induce in-growth of surrounding tissue which reduces migration.

According to some exemplary embodiments of the invention, a soft matrix comprising radioactive particles (e.g. micro-spheres) is injected to produce a non-migrating radioactive marker. In an exemplary embodiment of the invention, the soft matrix induces tissue-in-growth. Optionally, the soft matrix disperses or is resorbed within the body over time. In an exemplary embodiment of the invention, decay of the soft matrix does not produce migration of a physiologically significant amount of radio-isotope. The term “soft matrix”, as used in this specification and the accompanying claims indicates a material which does not harden and remains in an amorphous state.

In an exemplary embodiment of the invention, a soft matrix is characterized by a viscosity which is low enough to allow injection via a narrow gauge (e.g. 21, 23 or 26 g or narrower or intermediate diameters) needle but high enough to prevent undesired dispersal of micro-spheres contained in the soft matrix after injection. Optionally, the soft matrix is biodegradable and/or resorbable. In an exemplary embodiment of the invention, a collagen based (e.g. bovine collagen) based soft matrix is used as an amorphous mass. One example of a commercially available collagen based soft matrix suitable for use in the context of some embodiments of the invention is Artefill (Artes Medical, San Diego, Calif., USA).

In an exemplary embodiment of the invention, micro-spheres composed entirely of radioisotope containing material are injected in an amorphous mass to produce a non-migrating radioactive marker.

In some exemplary embodiments of the invention, a glue or cement based amorphous mass hardens to a solid mass. Optionally, the hardening reduces migration and/or dispersal of the micro-spheres.

In some exemplary embodiments of the invention, a soft matrix based amorphous mass is used that does not harden after injection. Optionally, a size of the micro-spheres prevents migration and/or dispersal. For example, micro-spheres with a diameter of 25 microns or more are resistant to phagocytosis by macrophages which might penetrate the soft matrix. Resistance to phagocytosis reduces a tendency towards dispersal and/or migration.

In an exemplary embodiment of the invention, the micro-spheres injected in the amorphous mass comprise a radioisotope coupled to another material (e.g. a cement or glue). Optionally, micro-spheres including a glue or cement are injected in a soft matrix amorphous mass. The radio-isotope can be coupled to the micro-spheres by any means known in the art, such as, for example, chemical bonding (e.g. covalent cross-linking or ionic bonding) and/or mechanical bonding, coating, or encapsulation.

In an exemplary embodiment of the invention, radioactive micro-spheres are coated or encapsulated with a non-radioactive material.

In an exemplary embodiment of the invention, the radioisotope is added to a micro-sphere production mixture prior to formation of the micro-spheres. For example, preparation of conventional non-radioactive PMMA micro-spheres might include combining a monomer component, a polymer component, a radio-opaque component (e.g. Barium Sulfate) and a catalyst component. Mixing conditions can be adjusted to produce spheres with desired properties. Addition of a radio-isotope in appropriate quantities to the reaction mixture can produce radio labeled PMMA micro-spheres with similar properties as their unlabeled counterparts. The activity/bead can be varied by adjusting the amount and/or specific activity of the added radio-isotope.

In an exemplary embodiment of the invention, the amorphous mass includes a material which induces tissue in-growth (e.g. calcium hydroxylapatite or calcium phosphate). The inducing material may be provided as part of the micro-spheres and/or between the micro-spheres (e.g. to prevent micro-sphere migration and/or bind the micro-spheres together). Optionally, tissue in-growth may be induced by mechanical and/or chemical properties of the material.

In other embodiments of the invention, a roughened surface of the micro-spheres contributes to tissue in-growth.

In an exemplary embodiment of the invention, the amorphous mass contains a gamma ray emitting material with an energy level and half life appropriate for use as a trackable permanently implanted marker. Optionally, the energy of the emitted gamma photons is 1000 kev, 500 kev, optionally 300 kev, optionally 100 kev or lesser or greater or intermediate values. Optionally, a half life of the gamma particle emitting material is in the range of 1-12 months, optionally 1-3 months or lesser or greater or intermediate times. These half lives are compatible with currently contemplated medical applications of the markers. Optionally, the energy level of gamma photon emitting material is selected in consideration of sensor geometry with lower energy levels being compatible with thinner sensors. 100-300 kev optionally provides useful sensor absorption and a medically acceptable level of body absorption.

In an exemplary embodiment of the invention, a detectable amount of radiation is supplied by the marker throughout a course of therapy while the amount of radiation from the marker persisting in the body after therapy is reduced. For example, if a 50 microCurie marker is desired for a 30 day medical protocol, a marker with an activity of 100 microCuries and a half life of 30 days might be implanted. Such a marker would have a 50 microCurie activity at the end of the protocol and 25 microCuries of activity 30 days after the protocol ended.

In an exemplary embodiment of the invention the amount of radiation is selected so that cytotoxicity is clinically insignificant throughout the life of the marker.

In an exemplary embodiment of the invention, radioactive micro-spheres are characterized by a size of 25, optionally 50, optionally 100 optionally 200 microns in diameter or lesser or intermediate or greater diameters. Optionally these sizes reduce phagocytosis.

### Exemplary Methods of Micro-sphere Production

In an exemplary embodiment of the invention, a quantity of radioisotope is mechanically bound to non-radioactive micro-spheres. Optionally, a population of micro-spheres is handled in a batch-wise or flow-through procedure. Mechanical binding may be achieved, for example, by dispersing the micro-spheres in a polymerization mixture containing a polymer (e.g. PMMA), a monomer (e.g. MMA) and a desired radioisotope. Optionally, spray drying yields micro-spheres with a radio-labeled PMMA polymer coating.

In an exemplary embodiment of the invention, a quantity of radio-active micro-spheres, or non-spherical micro-particles, (e.g. CO⁵⁷ micro-spheres) is coated with a non radioactive coating. The coating may be, for example, a glue or cement. In an exemplary embodiment of the invention, polylacticglycolic acid (PLGA) is used as the coating. PLGA is resorbable and/or promotes tissue in-growth. Optionally, 15 micron micro-spheres are coated with a 10 micron layer of PLGA to produce a coated micro-sphere with a diameter of 35 microns.

Optionally, the coated micro-spheres are subject to additional post-production processes such as polishing or roughening.

### Exemplary Solid Markers

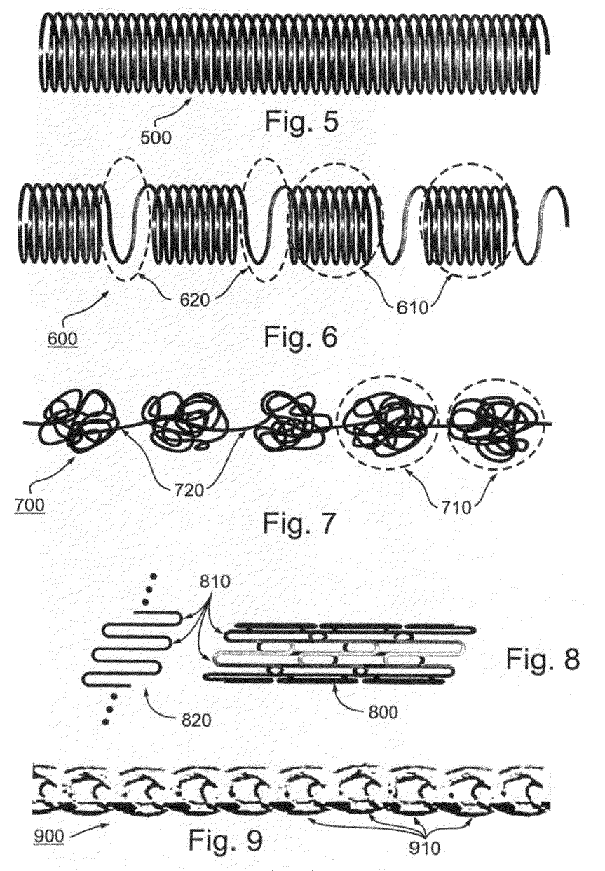

FIGS. 5, 6, 7, 8, 9, 10 and 11 illustrate different exemplary configurations of solid markers which constitute exemplary embodiments of the invention.

The goat of all of the embodiments illustrated in. FIGS. 5-13 is to construct a marker that contains sufficient radioactive and radio-opaque material so that it is detectable by a radiation detector and is visible in a medical image. In an exemplary embodiment of the invention, the marker is injectable through a narrow needle (e.g. 20, 21, 23, 27, 27 or narrower or intermediate gauges). Optionally, the marker has a pre-injection length of no more than 2-3 cm (for ease of injection). In an exemplary embodiment of the invention, the marker is flexible enough to crumple into a 2-5 mm diameter spherical or spheroid or ellipsoid shape upon injection into soft tissue.

FIG. 5 depicts an implantable marker 500 configured as a spring. Marker spring 500 can have a wide variety of different dimensions. In general, one or more of spring diameter, pitch and wire diameter can be adjusted in accord with an intended use of the marker. In an exemplary embodiment of the invention, marker spring 500 is at least partly constructed of wire containing a gamma emitting radioisotope.

Optionally, a diameter of spring 500 is in the range of 0.2-0.6 mm or lesser or greater or intermediate numbers of mm.

Optionally, a pitch of spring 500 is in the range of 0.01 mm and 0.2 mm or lesser or greater or intermediate numbers of mm.

Optionally, spring 500 is constructed from wire with a diameter in the range of 0.01-0.05 mm in diameter or lesser or greater or intermediate numbers of mm.

FIG. 6 depicts an exemplary implantable marker spring 600 which is similar to spring 500 except that it has alternating sections 610 and 620 with different pitches. Optionally, the tight sections 610 have a pitch between 0.01 mm and 0.2 mm or lesser or greater or intermediate numbers of mm. Optionally, sections 620 are loose and have a pitch between 0.2 and 1 mm or lesser or greater or intermediate numbers of mm. Loose sections 620 are spaced between tight sections 610 and optionally contribute to a tendency of marker spring 600 to deform into a spherical, spheroid or ellipsoid shape upon implantation in soft tissue. The lengths of sections 610 and/or 620 are optionally in the range of 0.1 mm to 1 mm or lesser or greater or intermediate numbers of mm.

FIG. 7 depicts an exemplary implantable marker 700 constructed from wire with a diameter in the range of 0.01-0.05 mm or lesser or greater or intermediate numbers of mm. Optionally, the wire contains a gamma emitting radioisotope. Portions of marker 700 are crumpled into “balls” 710. Optionally, each “ball” 710 is characterized by a diameter 0.2-0.6 mm or lesser or greater or intermediate numbers of mm. In the depicted embodiment “balls” 710 are separated by intervening sections 720 of un-crumpled wire which allow the marker to fold into a spherical shape upon implantation. Optionally, intervening sections 720 have a length of 0.1-1 mm or lesser or greater lengths. Optionally, “balls” 710 contribute to an increased visibility of marker 700 in a medical image. Optionally, the contribution to increased visibility comes from an appropriate density of metal for detection in a relevant imaging mode.

FIG. 8 depicts an exemplary implantable marker 800. Optionally, marker 800 is constructed from wire 0.01-0.05 mm or lesser or greater or intermediate numbers of mm in diameter. In an exemplary embodiment of the invention, the wire contains a gamma emitting radioisotope. In the depicted embodiment, the wire is folded back and forth at folds 810 to form a loose ribbon 820 which is wound helically into a cylindrical shape 800. Optionally, the cylindrical shape has a diameter of 0.2-0.6 mm or lesser or greater or intermediate numbers of mm. This exemplary configuration contributes to a degree of crushability in which a density of metal (from the wire) in marker 800 can be adjusted by modifying the density of the back and forth folds. Making the back and forth folds tighter together will increase the density while making them looser and farther apart with reduce the density.

FIG. 9 depicts an implantable marker 900 configured as a chain comprising links 910. Optionally, links 910 are characterized by a largest diameter of the link in the range of 0.2-0.6 mm or lesser or greater or intermediate numbers of mm. In an exemplary embodiment of the invention, marker 900 is constructed from material containing a gamma emitting radioisotope. This exemplary configuration provides crushability upon implantation and the ability to easily adjust the density of the metal in the marker by modifying the thickness of the wire used to form the links 910.

In an exemplary embodiment of the invention, an implantable marker includes a shield interposed between a source of gamma radiation in the marker and the surrounding tissue. In an exemplary embodiment of the invention, the shield selectively significantly inhibits beta radiation while having a minimal effect on gamma radiation. Optionally, this type of shield is also used with an amorphous marker, such as described above with reference to non-solid markers. Such a shield may optionally be integrally attached to the radioactive material, such as a in a platinum clad iridium core wire or other metal plated radioactive material, or it may optionally be an enclosure such as a plastic or metal tube within which the radioactive source is enclosed.

FIGS. 10, 11, 12a and 13a depict exemplary implantable markers with geometric structures compatible with coating a radioactive source with a layer of shielding material. Optionally, the layer of shielding material is characterized by a thickness of 0.025-0.25 mm or lesser, intermediate or greater thickness. Optionally, the layer of shielding material permits use of a gamma emitting radioisotope that also emits beta particles. Beta particles are typically absorbed within a few millimeters as they pass through soft tissue and therefore are ill suited for detection by a sensor placed outside the body. However, beta radiation can add significantly to total radiation dose absorbed by the patient from the implantable marker. Optionally, the shielding material can include any material which effectively absorbs beta particles, such as, for example, a plastic and/or a heavy metal. Heavy metals have an advantage over many plastics in that they can withstand being irradiated in a nuclear reactor, which allows the radioisotope to be activated after the shield is already incorporated into the marker. Appropriate selection of the shielding material will allow the marker to be activated after it is constructed, with any unwanted radioisotopes decaying much more rapidly than the radioisotope of interest.

One example of such a shielded marker uses Iridium-192 which has a half-life of 74 days. Shielding the Iridium-containing core with Platinum or Gold, and activating the marker in a nuclear reactor, would produce unwanted radioisotopes with half-lives on the order of minutes or hours which would decay to insignificant levels within 10-60 days while leaving the desired activity of Iridium-192.

FIG. 10 depicts an implantable marker 1000 configured as a wire with a maximum diameter of 0.2-0.6 mm or lesser or greater or intermediate diameters and containing a gamma emitting radioisotope. Marker 1000 is constructed to have alternating sections of different diameter 1010 and 1020. The narrow sections 1020 have a diameter between 0.01 and 0.1 mm or lesser or greater or intermediate diameters. The lengths of sections 1010 and/or 1020 can be 0.1 mm to 1 mm or lesser or greater or intermediate lengths. In an exemplary embodiment of the invention, the wire from which marker 1000 is constructed has a core which contains the radioisotope and an outer layer which is composed of shielding material. The ratio of the diameters of the core and shielding can be in the range of 0.1:1 to 10:1 or lesser or greater or intermediate ratios.

FIG. 11 depicts an implantable marker 1100 configured as a ball chain made of balls 1110. Optionally, balls 1110 are characterized by a diameter of 0.2-0.6 mm or lesser or greater or intermediate diameters. In an exemplary embodiment of the invention, balls 1110 contain a gamma emitting radioisotope and are connected by a wire 1120. Optionally, wire 1120 is characterized by a diameter of 0.01-0.1 mm or lesser or greater or intermediate diameters. Optionally, sections of wire 1120 are 0.1-1 mm in length or lesser or greater or intermediate length. Balls 1110 optionally have a core 1140 containing the radioisotope and an outer layer 1130 including shielding material. In an exemplary embodiment of the invention, the ratio of a diameter of core 1140 and shielding layer 1130 is in the range of 0.1:1 to 10:1 or lesser or greater or intermediate ratios. In an exemplary embodiment of the invention, the connecting wire is formed of the shielding material or of any other flexible biocompatible material.

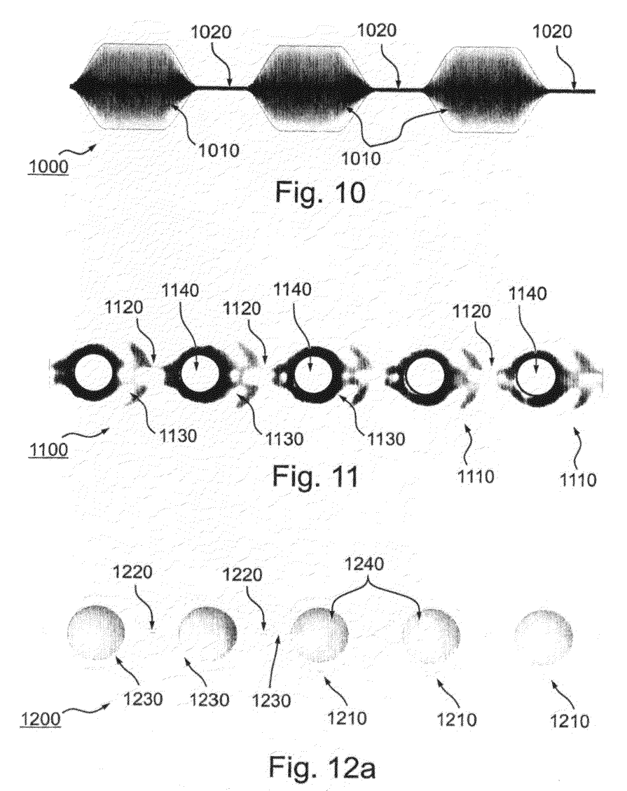

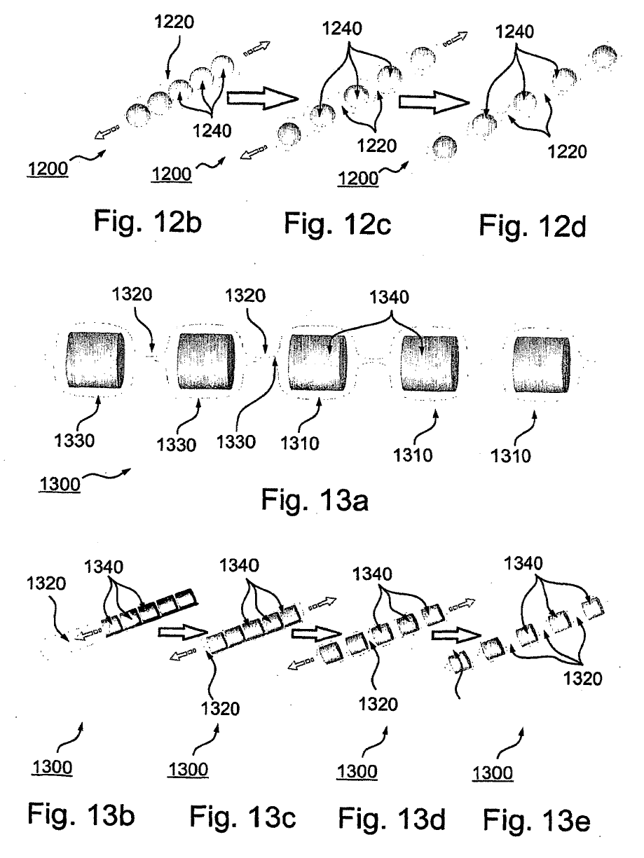

FIG. 12a depicts an implantable marker 1200 configured as a chain of segments 1210. In the depicted embodiment, each segment 1210 comprises a ball 1240 encased in a tube 1230, which is optionally shared by some or all the balls. Optionally, balls 1240 are characterized by a diameter of 0.2-0.6 mm or lesser or greater or intermediate diameters. In an exemplary embodiment of the invention, balls 1240 contain a gamma emitting radioisotope. In the depicted embodiment tube 1230 is characterized by constricted portions 1220 between the segments 1210. Optionally, constricted portions 1220 are characterized by a diameter of 0.01-0.1 mm or lesser or greater or intermediate diameters and a length of 0.1-1 mm or lesser or greater or intermediate lengths. Constriction can be achieved, for example by twisting or shrinking. Shrinking can be achieved, for example by stretching and/or by chemical treatment and/or by heat treatment. In an exemplary embodiment of the invention, balls 1240 include a radioactive core and an outer layer including shielding material. In an exemplary embodiment of the invention, tube 1230 includes shielding material. Optionally, a ratio of the diameters of a radioactive portion of ball 1240 and a thickness of shielding can be in the range of 0.1:1 to 10:1 (or lesser or greater or intermediate ratios). Optionally, both tube 1230 and balls 1240 include shielding material. In an exemplary embodiment of the invention, the radioactive material is Iridium-192, and a 0.1 mm thick layer of platinum absorbs all of the beta particles emitted by the Iridium-192 source.

FIG. 13a depicts an implantable marker 1300 similar to implantable marker 1200 with cylindrical beads 1340 instead of balls. Each segment 1310 comprises a bead 1340 within a sleeve 1330. Sleeve 1330 is characterized by constricted portions 1320 between cylindrical beads 1340. Other features, including optional features of marker 1300 are similar to marker 1200. In some manufacturing techniques cylinders are easier to manufacture than spheres since they can be cut from a continuous wire.

Exemplary Marker preparation Methods

Exemplary markers 1200 and 1300 depicted in FIGS. 12a and 13a respectively can be prepared in a variety of ways. FIGS. 12b, 12c, and 12d depict schematically an exemplary assembly sequence for exemplary marker 1200 as described hereinabove. FIGS. 13b, 13c, 13d and 13e depict schematically an exemplary assembly sequence for exemplary marker 1300 as described hereinabove.

FIG. 12b depicts balls 1240 inserted in sleeve 1230. Optionally, constrictions 1220 are produced between balls 1240 as illustrated sequentially in FIGS. 12c and 12d. According to various embodiments of the invention, constriction of sleeve 1230 is induced by stretching and/or heating and/or treatment with appropriate chemicals.

For example, a Teflon (manufactured by E.I. du Pont de Nemours) tube with an inner diameter of 0.25 mm and wall thickness of 0.1 mm can be filled with balls having a diameter of 0.2 mm and then stretched so that it shrinks, tightly grabs the balls, and pinches between them to a diameter of approximately 0.05 mm. The balls themselves can include a radioactive core including Iridium-192 with a diameter of 0.1 mm and a 0.05 mm thick layer of platinum shielding. Exemplary combination of the 0.05 mm shielding incorporated into the ball and the approximately 0.025 nm thickness of the tube significantly reduces the radiation dose received by the surrounding tissue from the beta radiation of the source.

FIG. 13b depicts cylindrical beads 1340 being inserted in a sleeve 1330. Optionally, cylindrical beads 1340 are constructed by slicing a wire. According to different exemplary embodiments of the invention, the wire is sliced completely or partially. Optionally, partial slicing contributes to an ease of insertion of beads 1340 in the sleeve since they remain connected until after they are inserted into the sleeve.

In the embodiment depicted in FIG. 13b, a wire which has been partially sliced is inserted in the sleeve. FIG. 13c depicts an end of the insertion process with beads 1340 still connected to one another. FIG. 13d shows beads 1340 separated from one another within the sleeve with constricted portions 1320 beginning to form. Optionally, separation is achieved by bending, rotating, or stretching the sleeve to break a connection between beads 1340. FIG. 13e depicts separation of beads 1340 by constricted portions 1320 formed, for example, by continued stretching (e.g. as described above) of the tube and/or by the application of heat and/or by the application of appropriate chemicals.

### Exemplary Radiation Sensors

In an exemplary embodiment of the invention, the implantable marker broadcasts its location radially outward as photons resulting from radioactive disintegrations. Optionally, a portion of this broadcast is received by one or more directional sensors deployed for that purpose. Exemplary sensors and corresponding performance data are described in co-pending. PCT applications WO 2006/016368 filed on Aug. 11, 2005 and entitled “Localization of a Radioactive Source within a Body of a Subject” and PCT/IB2006/052770 filed Aug. 10, 2006 and entitled “Localization of a Radioactive Source” the disclosures of which is incorporated herein by reference.

### Exemplary Considerations for Visibility in Medical Imaging

In an exemplary embodiment of the invention, the implantable marker is constructed and/or sized to be clearly visible in a medical image of one or more selected types (e.g. x-ray, fluoroscopy, CT, MRI and Ultrasound) Optionally, the marker does not induce significant streaking artifacts in CT images so that use of an implantable marker does not detract from a diagnostic and/or radiation therapy planning value of the image(s).

In order to achieve visibility without introducing undesired artifacts, there is generally a trade off between radio-opacity and the dimensions of the marker. In x-ray imaging (including CT and fluoroscopy), the more radiation the marker. absorbs the more clearly visible it is in the image so that use of a material with a higher absorption coefficient allows visibility of a marker with smaller dimensions.

On the other hand, in some imaging modalities (e.g. CT), too much absorption can cause artifacts (e.g. streaks).

In an exemplary embodiment of the invention, a marker that absorbs about two to ten times the amount of radiation as bone and is between 2 mm and 5 mm in diameter (absorbing between about 2% and about 25% of the radiation, depending on the energy level) provides an acceptable level of visibility in both x-ray and CT images. This physical configuration is typically not large enough to interfere in a clinically significant way with the soft tissue into which it is implanted.

Optionally, the implantable marker includes a non-solid material (e.g. gel, adhesive or cement). In those exemplary embodiments of the invention in which the marker includes a gel or cement matrix the radio-opacity of the marker can be controlled by modifying the volume of the non-solid material and/or by modifying its composition. For example, if a gel or cement matrix is not sufficiently radio-opaque; micro-spheres or particles of a radio-opaque material can be added in the appropriate quantities in order to adjust the radio-opacity of the marker using visibility considerations for x-ray and/or CT images as described above.

Optionally, the marker comprises primarily a metal body, such as a wire. In those embodiments of the invention in which the marker comprises a continuous metal material deployed in a tissue, a degree of radio-opacity can be adjusted by controlling a volume throughout which the marker is deployed and/or a percentage of the volume filled by the metal material. In an exemplary embodiment of the invention, the implanted marker defines a volume of 2 to 5 mm diameter (or intermediate diameters) within the soft tissue but physically occupies only 5, 10, 15, 25, 50 or 75% or lesser or intermediate percentages of the defined volume.

In an exemplary embodiment of the invention, the marker is composed of a coil which folds upon itself during implantation. Optionally, a density of metal within the coil is adjusted by adjusting one or more of wire diameter, coil diameter and pitch of the coil. Optionally, a percentage of the volume defined by the marker which is actually occupied by metal of the marker depends on one or more factors including, but not limited to, mechanical properties of the coil, the implantation tool, and properties of the tissue surrounding the marker. Interaction between these factors determines how the marker folds upon itself when released from the implantation tool. In an exemplary embodiment of the invention, all of these factors are considered when preparing a marker in order to achieve appropriate radio-opacity for x-ray and/or CT visibility.

Once markers have been produced according to theoretical considerations it is possible to test their actual behavior experimentally. Testing can be, for example, by injection into a block of gelatin with a density close to a density of a relevant soft tissue. Gelatin can be sufficiently transparent to allow visual assessment of marker configuration after injection. Optionally, marker configuration is observed for a period of time corresponding to a proposed implantation duration, or longer. Alternatively, or additionally, testing can be performed by injection in situ into a piece of a relevant soft tissue removed from a body. Optionally, animal tissue (e.g. rat, rabbit, chicken, pig, or dog) is used as a model for a corresponding human tissue. Alternatively, or additionally, testing can be performed in vivo using animals. In an exemplary embodiment of the invention, chicken liver serves as a model for human liver.

As a non-limiting example of theoretical marker design, an exemplary embodiment of the invention, in which a coil made of an alloy of 90% Platinum and 10% Iridium is fashioned into a marker is presented in detail. The exemplary Platinum/Iridium marker can be designed to have a desired level of radio-opacity according to the following procedure.

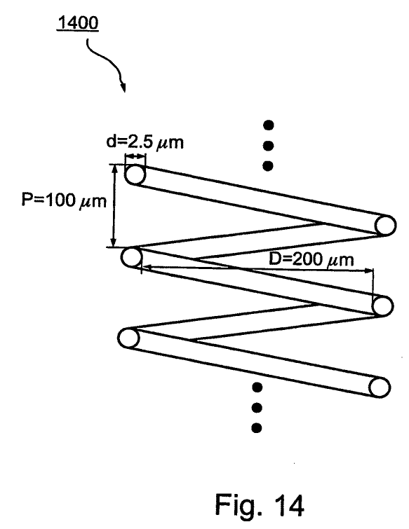

The metal percentage by volume of a coil made from wire with a diameter of 25 microns having an inner coil diameter of 200 microns and a pitch of 100 microns (FIG. 14 shows a coil with these exemplary dimensions) can be calculated as follows:

The total volume of a single turn of the coil is

$$\begin{matrix}π \times {( \frac{D + 2 d}{2} )}^{2} \times P = π \times {125}^{2} \times 100 \\ = 4908738 µ {m}^{3}\end{matrix} \qquad EQUATION 1$$

The length of wire in a single turn of the coil is

$$\begin{matrix}\frac{π \times ( D + d )}{cos ( arctan ( \frac{P}{π \times ( D + d )} ) )} = \frac{π \times 225}{cos ( arctan ( \frac{100}{π \times 225} ) )} \\ = 713.9 µm\end{matrix} \qquad EQUATION 2$$

The volume of wire in a single turn of the coil is therefore

$$\begin{matrix}713.9 \times π \times {( \frac{d}{2} )}^{2} = 713.9 \times π \times {12.5}^{2} \\ = 350435 {µm}^{3}\end{matrix} \qquad EQUATION 3$$

The metal percentage by volume is then calculated as the volume of the wire in a single turn of the coil divided by the total volume of a single turn of the coil

$$\frac{350435}{4908738} = 0.07 = 7 \% \qquad EQUATION 4$$

Such a coil has been found in in-situ animal studies to fold upon itself when slowly implanted through a standard 25 gauge biopsy needle into soft tissue, for example liver, so that the coil occupies about 10% of the volume throughout which it disperses. In this case, the metal percentage by volume of the implanted marker is therefore

7%×10%=0.07×0.1=0.007=0.7% EQUATION 5

The average path length through a sphere is 66.86% of its diameter. The average path length through a 5 mm diameter sphere is therefore 3.34 mm. The average photon passing through a 5 mm diameter sphere whose volume is 0.7% metal will therefore pass through

3.34 mm×0.007=0.023 mm of metal. EQUATION 6

The mass energy absorption coefficient, μₑₙ, of 10% Iridium/90% Platinum at 70 keV can be calculated as follows:

$$\begin{matrix}\frac{{μ}_{en}}{ρ} = {\sum }_{i}^{ } {{W}_{i} ( \frac{{μ}_{en}}{ρ} )}_{i} \\ = 0.1 \times 2.07 + 0.9 \times 2.14 \\ = 2.133\end{matrix} \qquad EQUATION 7$$

### Where:

Wᵢ is the percentage by weight of the i^{th} component (0.1 for Iridium and 0.9 for Platinum):

$$\frac{{μ}_{en}}{ρ}$$

### of Iridium at 70 keV is 2.07: and

$$\frac{{μ}_{en}}{ρ}$$

The density of 10% Iridium/90% Platinum is calculated by

$$\begin{matrix}ρ = \frac{1}{{\sum }_{i}^{ } {w}_{i} ( \frac{1}{{ρ}_{i}} )} \\ = \frac{1}{0.1 \times \frac{1}{22.42} + 0.9 \times \frac{1}{21.45}} \\ = 21.54\end{matrix} \qquad EQUATION 8$$

### Where:

ρ of Iridium is 22.42 g/cm³; and

ρ of Iridium is 21.45 g/cm³.

The μₑₙ, of 10% Iridium 90% Platinum is then found to be

$${μ}_{en} = ( \frac{{μ}_{en}}{ρ} ) \times ρ = 2.133 \times 21.54 = 45.94 \qquad EQUATION 9$$

The average percentage absorption of 70 keV photons passing through the marker can then be calculated as:

1−e^{-μ}^{ₑₙ}^{x}=1−e^{-45.94*0.0023}=0.1=10% EQUATION 10

where x is the total thickness of metal that the average photon passed through in centimeters. This is an average, based on the simplification of assuming that the metal in the marker is uniformly distributed throughout the volume occupied by the marker. This is a reasonable approximation given the large size of the pixels/voxels of the images relative to the size of the contortions of the crumpled marker.

In an exemplary embodiment of the invention, this percentage absorption can be increased or decreased as necessary produce a desired visibility in X-ray or CT images by adjusting the size of the marker. Optionally, size adjustment can be achieved by adjusting one or more of a length of the coil that is implanted, a thickness of the wire, a diameter of the coil and the pitch of the coil.

One of ordinary skill in the art will easily be able to modify calculations presented above to account for such adjustments in order to calculate a resulting absorption of the modified marker. Alternatively, or additionally, equations presented above, and others derived therefrom, can be used to perform an inverse optimization calculation in which a desired absorption characteristic is an input and one or more geometric parameters of the marker are calculated.

One of ordinary skill in the art will be able, using the above calculations as a guide, to develop parallel equations for markers with different geometries in order to calculate the percentage of radiation absorbing material within the volume of the marker and then to calculate the percentage of photons that will be absorbed. Alternatively or additionally, a numerical simulation may be used.

### Exemplary Radiation Parameters

In an exemplary embodiment of the invention, gamma energies in the range of 100-500 kev are high enough energy to escape the body, while being low enough energy to be captured efficiently by radiation detectors and not cause unnecessary radiation damage to the patient.

The level of radioactivity necessary to provide sufficient photons at the detectors depends on the properties of the selected radioisotope, including the number of photons emitted per decay and the energy of the emitted photons. For Iridium-192, for example, an activity in the range of 0.01-0.1 mCi provides the appropriate number of photons depending on the distance between the detectors and the patient and on the required tracking performance for the particular application.

In an exemplary embodiment of the invention, Iridium-192 is used in the implantable marker because it is already approved for use in medical applications and is generally considered safe to introduce into the body of a subject. However this isotope is only an illustrative example, and should not be construed as a limitation of the invention. When choosing an isotope for use in an implantable marker according to an exemplary embodiment of the invention, activity (DPM), type and energy of radiation and/or half life may be considered. It is generally desired that disintegration events be detectable with reasonable efficiency at the relevant distance, for example 20-50 cm. Long half lives may be preferred because they make inventory control easier and reduce total costs in the long run by reducing waste. However, short half lives may reduce concerns over radioactive materials and/or may allow smaller sources to be used.

### Exemplary Half Life Considerations

In an exemplary embodiment of the invention, the implantable marker includes Iridium (IR¹⁹²). Iridium is characterized by a half life of 73.8 days. According to exemplary embodiments of the invention, isotopes with a half life of 30, optionally 50, optionally 70, optionally 90 days optionally 270 days or greater or intermediate half lives are employed in the implantable marker. In an exemplary embodiment of the invention, these isotopes are compatible with a therapy regimen that lasts 2, optionally 4, optionally 8, optionally 10, optionally 12 weeks or lesser or intermediate or greater numbers of weeks. Exemplary therapies for which implanted trackable radioactive markers may be relevant include, but are not limited to, external beam radiation therapy, proton therapy, biopsy, laparoscopy, minimally invasive surgery, and robotic surgery.

### Safety

In an exemplary embodiment of the invention, the implantable marker is left in place at the end of the treatment. Optionally, an amount of radiation from the marker is low enough and/or a half life of an isotope included the marker is short enough that there is no significant danger to the patient.

In an exemplary embodiment of the invention, the marker is constructed of biocompatible materials. Optionally, the biocompatible materials are resorbable materials. Optionally, the biocompatible materials comprise an inert coating. Optionally, the inert coating reduces a tendency of the marker to elicit an immune or inflammatory response and/or reduces a tendency of marker components to disperse in the body.

### General

A variety of numerical indicators have been utilized to describe the implantable medical marker, radiation from the marker, radiation energy and/or relationships between the implantable marker and surrounding tissue. It should be understood that these numerical indicators could vary even further based upon a variety of engineering principles, materials, intended use and designs incorporated into the invention. Additionally, components and/or actions ascribed to exemplary embodiments of the invention and depicted as a single unit may be divided into subunits. Conversely, components and/or actions ascribed to exemplary embodiments of the invention and depicted as sub-units may be combined into a single unit with the described/depicted function.

Alternatively, or additionally, features used to describe a method can be used to characterize an apparatus (e.g. implantable marker) and features used to describe an apparatus can be used to characterize a method.

It should be further understood that the individual features described hereinabove can be combined in all possible combinations and sub-combinations to produce exemplary embodiments of the invention. The examples given above are exemplary in nature and are not intended to limit the scope of the invention which is defined solely by the following claims.

The terms “include”, “comprise” and “have” and their conjugates as used herein mean “including but not necessarily limited to”.

1. An implantable medical marker, the marker comprising:
(a) a marker body adapted for insertion via a needle and adapted to define a volume with a smallest dimension larger than an inner diameter of the needle; and
(b) a radiation source—characterized by gamma emissions sufficient to exit the human body.

2. A marker according to claim 1, wherein the smallest dimension is at least 1 mm.

3. A marker according to claim 1, wherein the gamma emissions produce between 1×10⁵ and 3×10⁸ photons/second.

4. A marker according to claim 1, wherein the gamma emissions produce not more than 5×10⁷ photons/second.

5. A marker according to claim 1, wherein the gamma radiation is characterized by an average energy of at least 50 kev.

6. A marker according to claim 1, wherein the gamma radiation is characterized by an average energy of at least 150 kev.

7. A marker according to claim 1, wherein the gamma radiation is characterized by an average energy not exceeding 400 kev.

8. A marker according to claim 1, wherein the gamma radiation is characterized by an average energy not exceeding 1000 kev.

9. A marker according to claim 1, wherein the marker is characterized by at least 1% absorption of an incident X-ray beam on the defined volume.

10. A marker according to claim 1, wherein the marker induces a radiation dose not exceeding 100 Gy at a distance of 2 mm from the marker in 6 months.

11. A marker according to claim 1, wherein the marker induces a radiation dose not exceeding 40 Gy at a distance of 2 mm from the marker in 6 months.

12. A marker according to claim 1, wherein the marker body includes one or more disorganized sections.

13. A marker according to claim 1, wherein the defined volume is only partially occupied by the marker body.

14. A marker according to claim 13, wherein the marker body is disorganized.

15. A marker according to claim 1, wherein a center of opacity and a center of radioactivity are both within the defined volume.

16. A marker according to claim 1, wherein a center of opacity and a center of radioactivity are in a predefined spatial relationship with respect to one another.

17. A marker according to claim 1, wherein a center of opacity and a center of radioactivity are spaced apart less than 20% of the largest dimension of the defined volume.

18. A marker according to claim 1, characterized by a spherically uniform distribution of radiation emission within 30%.

19. A marker according to claim 1, characterized in that at least a portion of the marker is adapted to absorb between 2 and 25 percent of 70 kev X-ray radiation incident on the marker.

20. A marker according to claim 1, wherein the marker body is adapted for insertion via a 21 gauge needle.

21. A marker according to claim 1, wherein the marker body is adapted for insertion via a 23 gauge needle.

22. A marker according to claim 1, wherein the marker body is a volume of non-solid material.

23. A marker according to claim 22, wherein the volume of non-solid material includes micro-spheres which promote tissue in-growth.

24. A marker according to claim 22, wherein the non-solid material is selected from the group consisting of a gel, a glue and a cement.

25. A marker according to claim 22, wherein the non-solid material is bio-absorbable.

26. A marker according to claim 22, wherein the radiation source comprises radioactive micro-spheres mixed into the non-solid material.

27. A marker according to claim 22, wherein micro-spheres are characterized by a degree of radio-opacity which contributes to a visibility of the marker in the X-ray based imaging mode.

28. A marker according to claim 22, wherein the volume of non-solid material includes micro-spheres which are characterized by a degree of radio-opacity which contributes to a visibility of the marker in the X-ray based imaging mode.

29. A marker according to claim 22, wherein the non-solid material includes the radiation source.

30. A marker according to claim 22, wherein the non-solid material is characterized by a degree of radio-opacity which contributes to a visibility of the marker in an X-ray based imaging mode.

31. An implantable marker according to claim 1, wherein the marker body is constructed of a radio-opaque radioactive metal.

32. An implantable marker according to claim 1, wherein the marker body comprises a radio-opaque radioactive wire with a spring-like memory.

33. An implantable marker according to claim 1, wherein the marker body comprises a coil.

34. An implantable marker according to claim 33, wherein the coil includes a Platinum/Iridium alloy.

35. An implantable marker according to claim 33, wherein the coil is adapted for folding.

36. An implantable marker according to claim 1, comprising a selective shield adapted to significantly reduce beta emissions from the marker.

37. A marker according to claim 36, wherein the selective shield comprises a material chosen from a heavy metal and a plastic.

38. A marker according to claim 36, wherein the selective shield comprises at least one material selected from platinum and gold.

39. A marker according to claim 36, wherein the selective shield is characterized by a thickness of 0.025 to 0.25 mm.

40. An implantable marker according to claim 31, wherein the marker body comprises a plurality of radioactive beads in a flexible sleeve.

41. A marker according to claim 40, wherein each radioactive bead includes a 0.025 to 0.250 mm thick layer of a beta radiation shielding material.

42. A marker according to claim 40, wherein the beads are characterized by a shape selected from among spherical and cylindrical.

43. A marker according to claim 1, wherein the marker body comprises a chain of beads connected by flexible wire.

44. A marker according to claim 43, wherein the beads comprise a radioactive radio-opaque material.

45. A marker according to claim 43, wherein the beads comprise a radio-opaque material surrounding a radioactive core.

46. A marker according to claim 43, wherein a radio-opaque material and the radiation source are each encapsulated within the beads.

47. A marker according to claim 43, wherein the beads comprise a beta shielding material adapted to selectively dampen beta emissions from the radiation source.

48. A marker according to claim 43, wherein the beads are characterized by a thickness of 0.025 to 0.25 mm.

49. A kit comprising a marker according to claim 1 and an insertion tool.

50. A kit according to claim 49, comprising an ejection tool.

51. A kit according to claim 49, wherein the insertion tool comprises a needle.

52. A kit according to claim 49, wherein the implanted marker is provided loaded into the insertion tool.

53. A method of preparing an implantable medical marker, the method comprising:
(a) inserting a plurality of radioactive beads into a sleeve; and
(b) inducing the sleeve to contract.

54. A method according to claim 53, wherein the inducing includes at least one action selected from the group consisting of stretching, heating and chemically treating the sleeve.

55. A method according to claim 53, wherein the contraction forms narrowed portions between the beads.

56. A method of preparing an implantable medical marker, the method comprising:
(a) associating an amount of gamma radiation with a metal wire;
(b) forming the wire into a desired shape; and
(c) inserting the wire in a needle.

57. A method of producing a non-migrating radioactive marker in situ in a subject, the method comprising:
(a) preparing an aliquot of radio-labeled microspheres; and
(b) injecting the aliquot at a desired location.

58. An injectable pharmaceutical composition for formation of a non-migrating radioactive implant, the pharmaceutical composition comprising:
(a) an active ingredient including an aliquot of radioactive microspheres, and
(b) carriers, diluents and excipients.

59. An implantable medical marker; comprising:
(a) a plurality of radioactive microspheres; and
(b) a biocompatible amorphous mass including said microspheres;
wherein the amorphous mass is adapted to prevent dispersion of the radioactive microspheres within the body.

60. A method according to claim 1, wherein the marker body is adapted to remain contiguous.
